(19) United States
(12) Patent Application Publication
(10) Pub. No.: US 2002/0098242 A1
(43) Pub. Date: Jul. 25, 2002

(54) Oral pharmaceutical preparation comprising an antiulcer activity compound, and process for its production

(76) Inventor: Carlos Picornell Darder, Madrid (ES)

Correspondence Address: THOMAS C. PONTANI, ESQ., COHEN PONTANI LIEBERMAN & PAVANE, 551 FIFTH AVENUE, SUITE 1210, NEW YORK, NY, 10176, US

(21) Appl. No.: 09/491,624

(22) Filed: Jan. 26, 2000

(30) Foreign Application Priority Data
Jul. 13, 1998 (ES) PCT/ES98/00204
Jan. 27, 1999 (ES) P 9900157

(51) Int. Cl. A61K 9/16; A61K 9/50

(52) U.S. Cl. 424/490

(57) ABSTRACT

Disclosed is a pharmaceutical preparation and a process for making the same. The preparation has an inert nucleus; an active layer which is soluble or disintegrates rapidly in water, obtained from a single aqueous or hydroalcoholic solution-suspension which includes: an active ingredient of anti-ulcerous activity of formula I, II or III, and at least one excipient; and a gastro-resistant outer coating layer obtained from a solution which includes an enteric coating polymer and at least one excipient. The process is conducted by coating the inert nuclei by spraying a single aqueous or hydroalcoholic suspension-solution onto the nuclei; drying of the active layer formed during the spraying; and coating the charged nuclei by spraying of a solution which includes an enteric coating polymer with at least one excipient in order to obtain a gastro-resistant outer coating layer.

### CROSS REFERENCE TO RELATED APPLICATIONS

This application is a Continuation-in-Part of International PCT Application No. PCT/ES98/00204 filed Jul. 13, 1998 designating the United States of America.

### BACKGROUND OF THE INVENTION

1. Field of the Invention

The present invention relates to a new pharmaceutical formulation for oral administration which includes a compound of anti-ulcer activity, and to a procedure for making same.

2. Description of the Related Art

Numerous techniques recently have been developed for preparing systems of release in the form of microgranules wherein the mixture of active ingredient and excipients is submitted to a process of kneading, extrusion, spheronization, coating, etc. Each of these pelletization techniques calls for a different technology, so that there are many types of pelletization equipment, coating pans or drums, fluid-bed equipment, extruders-spheronizers and centrifuging equipment, among others. The final result would appear to be the same, although there are in fact considerable differences between the pellets made using each technique.

Various types of microgranules have been described for the formulation of certain benzimidazoles with anti-ulcer activity, such as those of European patents ER 247983, ER 244380, ER 237200 and ER 277741 and international patent WO 92/22284. This type of compound is in general acid-labile and for that reason various procedures have been developed to protect them from the effect of the gastric acid medium.

In European patents ER 247983 and ER 244380 the active ingredient is kneaded by a wet process with a mixture of excipients which allows an alkaline microenvironment to be created. The mixture is extruded and then spheronized. The spheronized microgranules are coated with one or more intermediate layers of excipients soluble in water, alkalis, buffer solutions, polymeric solutions, etc., and an external gastro-resistant layer is then applied.

As this is an extrusion-spheronization method, the total yield of the process will depend upon many factors. On the one hand, during the extrusion phase it is essential to control dimensions such as the cross-section and the length of the extrudate so as to avoid great dispersion in the size and shape of the particles. Both factors would explain the subsequent coating being irregular and would even lead to the presence of pores, unless an excess quantity were projected in order to ensure complete coating of the microgranule, though this would in turn cause problems when it came to standardizing release of the active ingredient. On the other hand, the characteristics of cohesiveness, firmness and plasticity of the extrudate must be controlled if its subsequent spheronization is to be ensured.

To these problems is added the fact that the need to use several pieces of equipment such as kneading machines, extruding machines and spheronizers means that losses through kneading, extrusion and spheronization can be greater than with other pelletization methods. European patents EP 237200 and EP 277741, this last published in Spain as ES 2.052.697, show an example of coating with sprinkled powder (powder-layering) using a rotogranulating machine. Spherical granules are described which have a nucleus coated with dusted powder which contains an anti-ulcer benzimidazolic compound and hydroxypropyl cellulose with low degree of replacement. Also described is a procedure for producing the aforesaid spherical granules, characterized in that the seeding nuclei are wetted by spraying thereof with an agglutinant solution and they are dusted with a powder which contains the active ingredient and the hydroxypropyl cellulose little replaced.

The technique of coating using a rotogranulating machine is very abrasive, especially in the initial phase of the process. Apart from abrasion of the particles against the walls of the machine due to the thrust of the air, a situation normal in any fluid bed, there is a shear force exercised by the rotary disc of the rotogranulating machine. All this often leads to problems such as breakage and abrasion of the granules.

These problems not only make control of the release of active ingredient more difficult, but also have a considerable effect on granule production output. For this reason, and in order to reduce these problems, European patent EP 277741 proposes as a solution the use of extremely hard seeding nuclei.

For the preparation of the aforesaid spherical granules, European patent 277741 describes the use of rotogranulating machine of centrifugal type such as the CF360 rotogranulating machine by Freund Co. In this procedure, two layers are added successively, though leaving them perfectly separate. In the first, the active ingredient is added with excipients in powder form simultaneously with a solution of the aqueous binder. In the second, the excipients are simply added in powder form along with the aqueous binder solution. The procedure of addition of the active layer according to EP 277741 means that the layer is quite porous and is distributed in a manner which is not perfectly uniform over the surface of the initial inert particle.

The spherical granules obtained are dried for sixteen hours and then passed through a cascade of sieves in order to select the best range of sizes. Finally, to apply the enteric coating, the dry sieved granules are placed in a “Wurster” type fluid bed. In short, the spherical granules with gastro-resistant coating described in European patent EP 277741 have passed through four different pieces of equipment.

### BRIEF DESCRIPTION OF THE DRAWINGS

In the drawings:

FIG. 1 is a photograph obtained by electron microscope scanning, showing a section of the ansoprazol pellet of Example 1;

FIGS. 2 and 3, are photographs also obtained by electron microscopy, showing further details of the layers present;

FIG. 4 is a photograph showing the porosity of the coating;

FIGS. 5, 6 and 7, are photographs showing a section of the omeprazol pellet of Example 2 with a gastro-resistant coating of formula I; and

FIG. 8 is a photograph showing the homogeneity of the coating and the few pores of same.

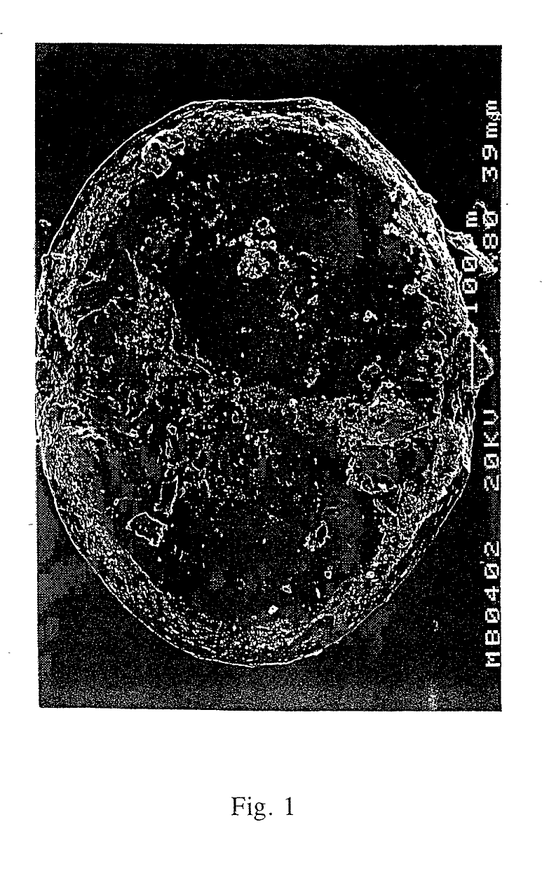

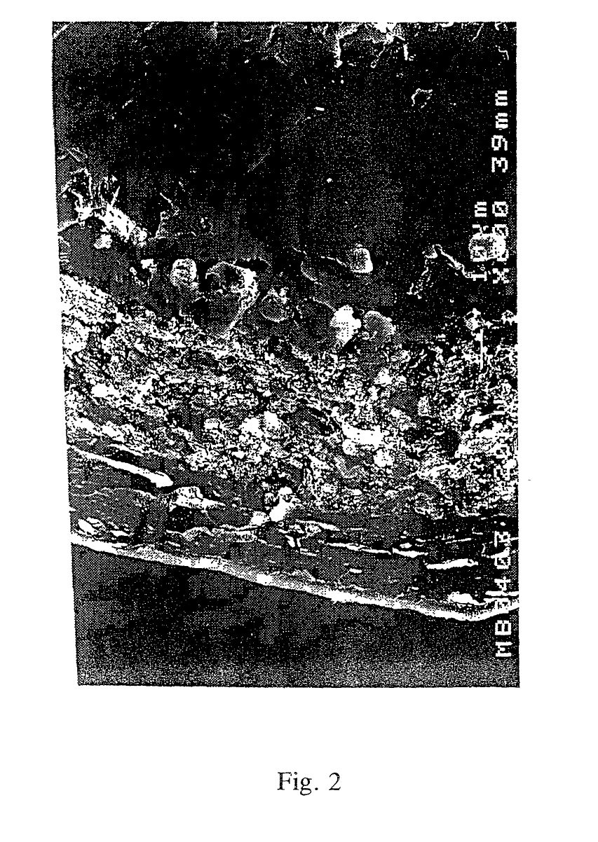

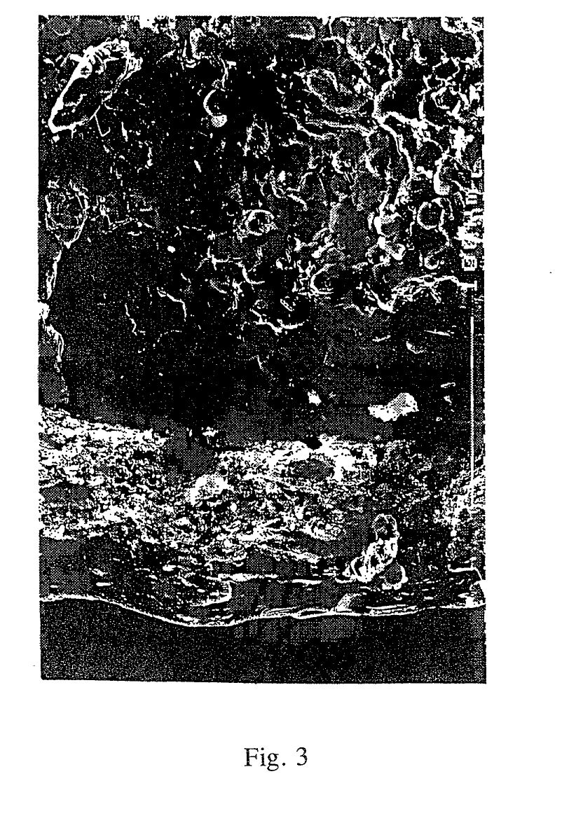

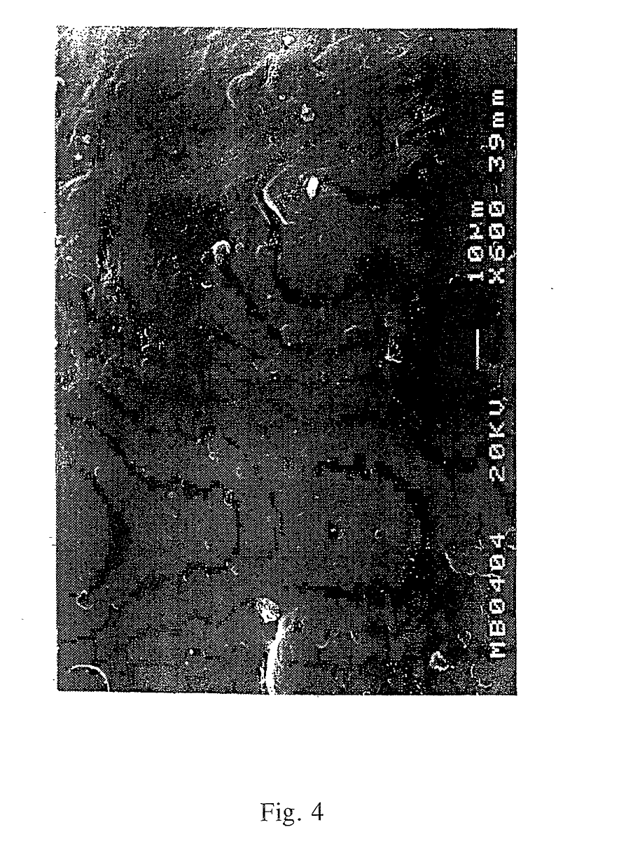

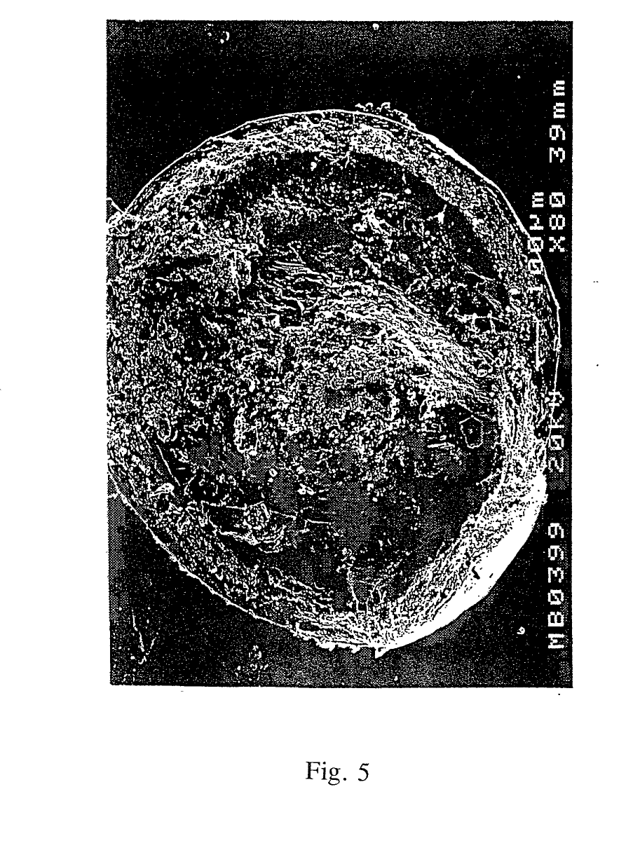

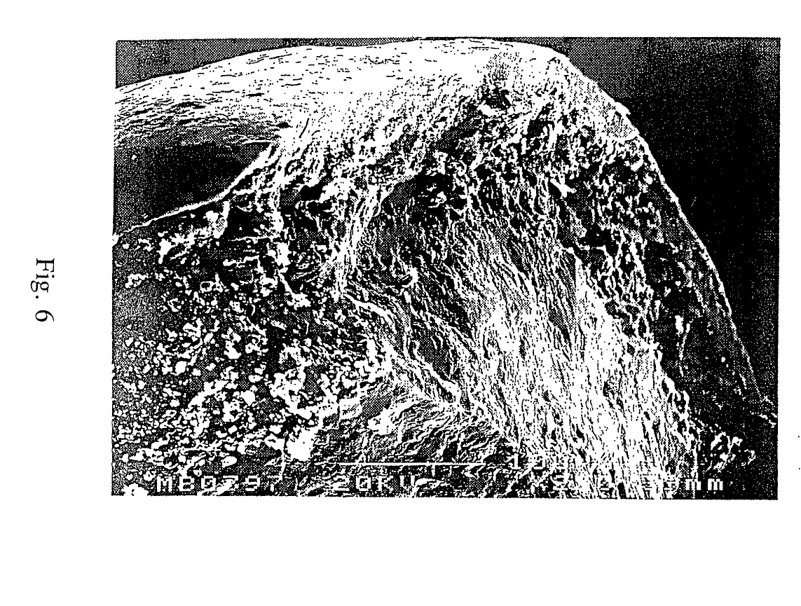

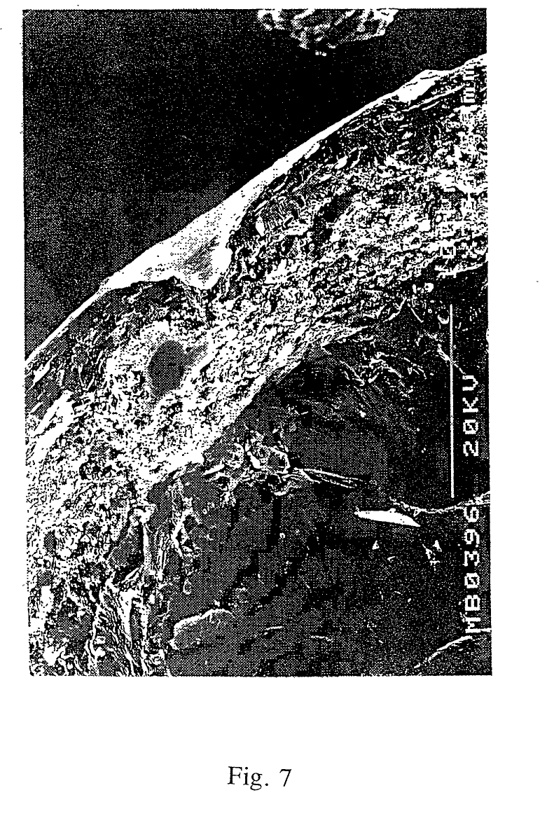

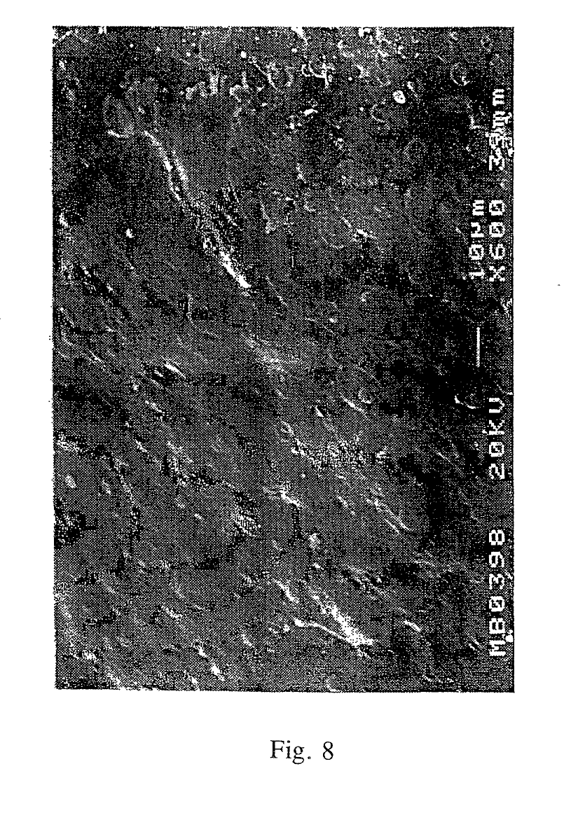

### DETAILED DESCRIPTION OF THE PRESENTLY PREFERRED EMBODIMENTS

In the present invention a formulation and a working methodology in a fluid bed of the “Wurster” type or the like have been developed. In it, the negative factors which affected the methods described to date are eliminated and substantial changes introduced with respect to the methods of previous patents for pellets containing benzimidazoles.

The object of the present invention is to find new pharmaceutical formulations for the oral administration of anti-ulcer active ingredients of the benzimidazole formula I type

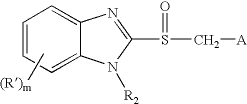

in which:

A can be:

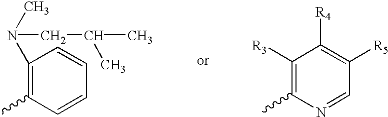

in which: R³ and R⁵ are the same or different, and can be hydrogen, alkyl, alkoxy, or alkoxyalkoxy; and

R⁴ is hydrogen, alkyl, alkoxy which can optionally be fluorated, alkoxyalkoxy, or alkoxycycloalkyl;

R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulphinyl;

R² is hydrogen, alkyl, acyl, carboalkoxy, carbamoyl, alkylcarbamoyl, dialkylcarbamoyl, alkylcarbonilmethyl, alkoxycarbonilmethyl or alkylsulphonyl; and, m is a whole number from 0 to 4;

or formula II or III:

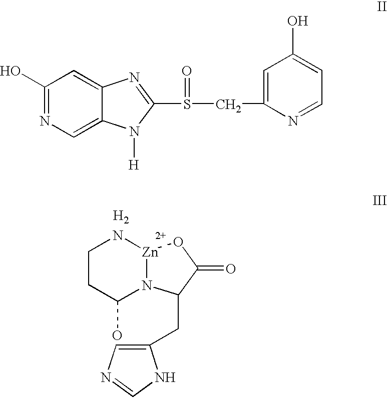

hereinafter generally denominated anti-ulcer compounds.

The new galenical formulations object of the present invention are characterized in that they are spherical granules with a homogeneous active charge layer and a very unporous surface, formed by coating of an inert nucleus by spraying a single aqueous or hydroalcoholic mixture containing the active ingredient (anti-ulcer compound) together with the other excipients. Then, in the same equipment and following a short drying period, the granules obtained are subjected to a stage of enteric coating. Optionally, if it is desired to obtain lower humidity, recourse can be had to additional drying.

Said formulations resolve satisfactorily and innovatively the difficulties described in the prior state of the art, while at the same time showing resistance to dissolution in acid medium (gastro-resistant) and dissolving rapidly in alkaline medium with disintegration of the granules and excellent release of active ingredient.

The present invention satisfactorily resolves the difficulty involved in coating the inert nucleus with an aqueous or hydroalcoholic solution suspension containing a un anti-ulcer compound which is generally highly labile in an acid environment or environment and in aqueous dissolution, in the presence of disintegrating-swelling excipients which cause an increase of viscosity which enormously hinders spraying thereof onto the inert nuclei.

El “Wurster” type fluid bed or the like in which the coating process is carried out minimizes the abrasion caused by rotogranulation. It is therefore unnecessary to use a specially hard inert nucleus.

The microgranule is not subjected to any kneading or extrusion process, nor is an inert nucleus coat sprinkled with powder dusted together with an aqueous binder. The microgranule used in the present invention consists in an inert nucleus which is coated with a single active layer made up of an aqueous or hydroalcoholic suspension-solution which includes the anti-ulcer component and at least one disintegrating-swelling excipient, a binder, an alkalizing medium, a surface-active agent and a diluent.

When a single suspension-solution is projected onto the inert nucleus, a less porous and more homogeneous product is obtained than in the procedures known to date, and all the subsequent operations are simplified considerably.

Likewise, unlike what happened in the prior art (EP 244.380, EP 277.983, EP 237.200, EP 277.741, PCT W092/22289), in which the manufacturing procedure was carried out using several different pieces of equipment, in the present invention the entire process is carried out using a single piece of fluid-bed equipment, thereby minimizing losses of time and of product, while more easily complying with Good Manufacturing Practice (GMP) for medicaments. What is more, avoidance of handling and intermediate steps considerably reduces the investment required in machinery and buildings.

The inert nuclei used are microspherical neutral granules which can have in their composition two or more of the following substances: sorbitol, manitol, saccharose, starch, microcrystalline cellulose, lactose, glucose, trehalose, maltitol and fructose. The initial size of same can be between 200 and 1800 micrometers, preferably between 600-900 micrometers.

The single aqueous or hydroalcoholic solution-suspension which is sprayed onto the inert nucleus is made up of the active ingredient with anti-ulcer activity and the other excipients. The hydroalcoholic medium is made up of mixtures of water:ethanol in proportions less than or equal to 50% v/v₁ preferably between 25% -45% v/v.

The oral pharmaceutical preparation of the present invention includes a compound with anti-ulcer activity as its active ingredient and is characterized in that it also includes:

a) an inert nucleus;

b) a soluble active layer or layer which disintegrates rapidly in water, made from a single aqueous or hydroalcoholic solution-suspension which includes:

an active ingredient of anti-ulcer activity of general formula I

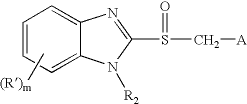

in which:

A can be:

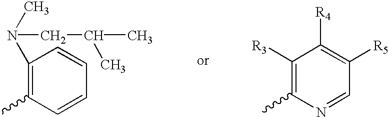

in which: R³ and R⁵ are the same or different, and may be hydrogen, alkyl, alkoxy, or alkoxyalkoxy; and

R⁴ is hydrogen, alkyl, alkoxy which can be fluorated, alkoxyalkoxy, or optionally alkoxycycloalkyl;

R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulphinyl;

R²is hydrogen, alkyl, acyl, carboalkoxy, carbamoyl, alkylcarbamoyl, dialkylcarbamoyl, alkylcarbonilmethyl, alkoxycarbonilmethyl or alkylsulfonil; and, m is a whole number from 0 to 4;

or of formula II or III,

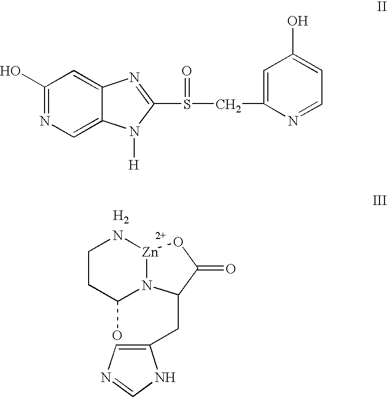

and

at least one pharmaceutically acceptable excipient selected from the group which includes: a binder, an alkaline reaction compound, a surface-active agent, a filling material and a disintegrating-swelling excipient; and

c) a gastro-resistant outer coating made from a solution which includes:

an enteric coating polymer; and

at least one excipient chosen from the group which includes: a plasticizer, a surface-active agent, a pigment and a lubricant.

Among the excipients present in the suspension-solution of the active compound of formula I, II or III which is sprayed onto the inert nuclei are:

a) a binder or mixture of binders: saccharose, starch, methyl cellulose, carboxymethyl cellulose (CMC), hydroxypropyl cellulose (HPC), hydroxypropilmethyl cellulose (HPMC), polyvinyl pyrrolidone (PVP), dextrine or gum arabic, dissolved in water, ethanol, or a mixture of both (50% v/v or less)

b) a compound with alkaline reaction, such as trisodium and disodium phosphate, the oxide, hydroxide or carbonate of magnesium, hydroxide of aluminium, carbonate, phosphate or citrate of aluminium, calcium, sodium or potassium, the mixed compounds of aluminium/magnesium Al₂O₃. 6MgO.CO₂. 12H₂O or MgO.Al₂O₃.2SiO₂.nH₂O or similar compounds and amino acids with alkaline reaction such as sodium, potassium, aluminum or calcium acetate; sodium, potassium, aluminum or calcium glycerophosphate; (tris)-hydroxymethylaminemethane (tromethamine); N-methylglucamine, 2-amine-2-methyl-1,3-propanediol; 2-amine-2-methyl-1propanole; sodium, potassium, magnesium, calcium, aluminum or aluminum hydroxide salts of aminoacids like lysine, glutamic acid, glycine or pyrimidinecarboxilic acids, like nicotinic acid, salts derived from organic or weak inorganic acids and bases like guanidine and basic aminoacids like arginine, histidine, lysine and triptophane.

c) a surface-active agent, such as sodium lauryl sulphate, polysorbate, poloxamer and ionic and non-ionic surface-active agents.

d) a filling material such as lactose, starch, saccharose, mannitol, sorbitol, gelatin or microcrystalline cellulose

e) a disintegrating-swelling compound, such as starch, calcium carboxymethyl cellulose (CMCCa), sodium glycolate starch or hydroxypropyl cellulose (L-HPC).

Once the microgranules have been formed by spraying the aqueous or hydroalcoholic suspension-solution containing the active ingredient, they are dried and coated with a layer of the enteric coating.

The following can be used as enteric coating polymers: methyl cellulose, hydroxyethyl cellulose (HEC), hydroxybutyl cellulose (HBC), HPMC, ethyl cellulose, hydroxymethyl cellulose (HMC), HPC, polyoxyethylene glycol, castor oil, cellulose phthalic acetate, phthalate of HPMC, succinate acetate of HMC, sodium carboxymethylamylopectin, chitosan, alginic acid, carrageenans, galactomannons, tragacanth, shellac, agar-agar, gum arabic, guar gum and xanthan gum, polyacrylic acids, methacrylics and their salts, HPMC acetate succinate, polyvinyl acetate phthalate, cellulose acetate trimethylate, polyvinyl alcohol (PVA), polyethylene and polyproprylene oxides and mixtures thereof. The gastro-resistant polymer can be accompanied by: plasticizers such as triethylcitrate (TEC), polyethylene glycol (PEG), diethyl phthalate, dibutyl phthalate, dimethyl phthalate, diocytl adipate, dioctyl phthalate, dioctyl tercphthalate, butyloctyl phthalate, triethylene glycol di-2-ethylhexanoate, trioctylmethylate, glyceryl triacetate, glyceryl tripropionate, 2,2,4-trimethyl-1,3-pentanedioldiisobutyrate, cetyl and stearyl alcohol; surface-active agents such as sodium lauryl sulphate, polysorbate and poloxamer; pigments such as titanium dioxide, iron sesquioxide; lubricants such as talc, magnesium stearate or glyceril monostearate, together with a mixture of same.

Another object of the present invention is a manufacturing procedure for said galenical formulations.

The procedure for obtaining the oral pharmaceutical preparation of the invention is as follows:

1) coating of the inert nuclei by spraying of a single aqueous or hydroalcoholic suspension-solution, described above, which includes:

the active ingredient of anti-ulcer activity of I, II or III, and

at least one pharmaceutically acceptable excipient selected from the group which includes: a binder, an alkaline reaction compound, a surface-active agent, a filling material and a disintegrating-swelling excipient;

2) drying of the active layer formed during the spraying of the previous stage to form charged nuclei; and

3) coating of the charged nuclei by spraying a solution which contains an enteric coating polymer with at least one pharmaceutically acceptable excipient selected from the group which includes: a plasticizer, a surface-active agent, a pigment and a lubricant, in order to form an gastro-resistant external coating layer.

Optionally, after stage 3) of coating of the charged nuclei, an additional drying is carried out.

There follows a description of the procedure of the invention, with special reference to the method and percentages used for each of the components.

In a tank of suitable dimensions an alkaline aqueous or hydroalcoholic solution is prepared by incorporating the alkaline-reaction compound into the aqueous or hydroalcoholic vehicle in a percentage of between 0.1%-5% (p/p). Using continuous agitation, the anti-ulcer benzimidazolic compound and another compound with anti-ulcer activity (6%-25% p/p) are incorporated together with the filler material (3-15% p/p). To the suspension-solution obtained is added the surface-active agent (0.01% -3% p/p), a binder and a disintegrating-swelling agent in percentages of between 2%-10% respectively, taking account of the times of use of the prepared solution.

Homogenization of the mixture is carried out with continuous agitation and at ambient temperature (23±2° C.) Agitation is maintained during the spraying phase of the active layer on the inert pellets; this process is carried out using a “Wurster” type fluid bed or similar equipment, into which the inert nuclei of size 850 Am are poured. The spraying conditions are as follows: Spraying pressure: 2-3 bar. Product temperature: 35-45° C. Volume of air: 700-1200 m³/h at 80-90° C. Nozzle diameter: 1.2 mm).

Once the charging phase has been completed, the nuclei coated with the active ingredient are dried in the same equipment. The air flow is 600-800 m³/h at temperature of 35-45° C. for 45 minutes.

The next stage is enteric coating of the active pellets, which is carried out in the same equipment. An aqueous or organic dispersion of the gastro-resistant polymer (10-40% p/p) is prepared. The plasticizer (0.2-10% p/p) is in turn dissolved in water and the surface-active agent added with constant agitation (up to 3% p/p) and, where necessary, pigments (0-5% p/p) and lubricants (0.5-16% p/p). Once the mixture has been homogenized the dispersion of the gastro-resistant polymer (25-45% p/p) is added whilst agitating.

In order to obtain lower humidity content, an additional drying can be carried out using a conventional dryer.

Over 90% of the resulting microgranules must be of a diameter between 0.4 and 1.95 mm, and more specifically between 0.5-1.8 mm.

The nuclei object of the present invention are resistant to dissolution in acid medium, dissolve rapidly in alkaline medium, are stable over long storage periods, have excellent disintegration characteristics, and the active layer is more homogeneous and less porous than the granules described in the previous patents.

The present invention resolves satisfactorily the disadvantages deriving from the prior art, since a single suspension-solution is prepared for charging the inert nuclei. For this phase a fluid bed of the Wurster type or the like is used, this being much less abrasive than the rotogranulating machine which has to be used when a seeding nucleus is coated with an active powder and a binder solution.

From the time that charging of the inert nuclei starts until the enteric coating is completed, the entire procedure is carried out on a single “Wurster” type fluid bed or the like, unlike other procedures which take place on several different pieces of equipment. For a better understanding of all that has been set out above, some examples are provided which, schematically and solely by way of non-restrictive example, show a practical example of embodiment of the invention.

### EXAMPLES

### Example 1

In a stainless steel receptacle of sufficient capacity an alcalizing aqueous solution of triosodium phosphate was prepared, and to this were added lansoprazol, lactose and sodium lauryl sulphate, with continuous agitation throughout. When the mixture was homogeneous the colloidal aqueous solution of hydroxypropylmethyl cellulose (13.50% p/p) was added, maintaining agitation in order to ensure homogeneity of the product. L-HPC was then incorporated into that solution-suspension. Agitation was maintained up till the moment of spraying onto the neutral pellets.

| | | |
|---|---|---|
| Lansoprazol | 1.29 | Kg |
| Sodium lauryl sulphate | 5.28 10⁻³ | Kg |
| Chrystallized disodium phosphate | 0.052 | Kg |
| Hydroxypropylmethyl cellulose | 0.8 | Kg |
| Lactose | 0.51 | Kg |
| Hydroxypropyl cellulose | 0.39 | Kg |
| Water | 14.28 | Kg |

10 kg of inert nuclei were incorporated, made up of saccharose (62.5-91.5%) and starch (37.5-8.5%) of 800 micrometers average size in a NIRO “Wurster” type fluid bed and was covered with the solution-suspension prepared in advance, under the following conditions: air flow: 250 m³/hr. Diameter of nozzles: 1.2 mm. Spraying pressure: 2.5 bar. Spraying of product: 100 g/min. Air temperature: 85° C. Product temperature: 38° C.

The charged nuclei were then dried in the same bed for 45 minutes with air at a temperature of 35° C. and with an air flow of 250 m³/h in order to obtain a suitable degree of humidity.

The dry granules were subjected to enteric coating by spraying the gastro-resistant solution-suspension detailed below and prepared from an aqueous solution of polyethylene glycol into which were incorporated the other excipients, with continuous agitation

| | | |
|---|---|---|
| Talc | 0.57 | Kg |
| Titanium dioxide | 0.18 | Kg |
| Polyethylene glycol 6000 | 0.18 | Kg |
| Polysorbate | 0.08 | Kg |
| Eudragit L30D55 | 5.78 | Kg |
| Water | 12.14 | Kg |

The working conditions were as follows: air flow: 250 m³/hr. Diameter of nozzles: 1.2 mm. Spraying pressure: 2.5 bar. Spraying of product: 100 g/mm. Air temperature: 70° C. Product temperature: 36° C.

Optional drying of the coated pellets was carried out for 45 minutes with air at a temperature of 35° C. and with an air flow of 250 m³/hr.

Set out below are the results of the stability studies carried out on a batch of Lansoprazol pellets under different storage conditions: ambient temperature, and 40° C. and relative humidity 75%.

| | | | | | | |
|---|---|---|---|---|---|---|
| | | Gastro- | | | | Transmittance |
| Test time | Colour | resistence | Release | Active Ing. | Humidity | at 440 nm |

Storage conditions: Ambient temperature
Container: Topaz glass bottle with bag of silica gel inside fitted with
metallic screw-threaded top including zelelastic seal

| | | | | | | |
|---|---|---|---|---|---|---|
| Zero hour | Cream | 98.8% | 82.8% | 33.O mg/370 mg | 1.62% | 97% |
| | white | | | | | |
| 1 month | Cream | 98.6% | 82.0% | 33.O mg/370 mg | 1.60% | 97% |
| | white | | | | | |
| 3 months | Cream | 97.0% | 80.9% | 32.S mg/370 mg | 1.48% | 97% |
| | white | | | | | |
| 6 months | Cream | 97.4% | 79.8% | 32.O mg/370 mg | 1.47% | 96% |
| | white | | | | | |
| 18 months | Cream | 97.4% | 78.9% | 31.9 mg/370 mg | 1.46% | 95% |
| | white | | | | | |

Storage conditions: Temperature: 40° C., 75% of humidity
Container: Topaz glass bottle with bag of silica gel inside fitted with
metallic screw-threaded top including zelelastic seal

| | | | | | | |
|---|---|---|---|---|---|---|
| Zero hour | Cream | 98.8% | 82.8% | 33.O mg/370 mg | 1.62% | 97% |
| | white | | | | | |
| 1 month | Cream | 97.8% | 81.2% | 32.O mg/370 mg | 0.90% | 95% |
| | white | | | | | |
| 3 months | Cream | 97.6% | 80.8% | 31.8 mg/370 mg | 1.27% | 93% |
| | white | | | | | |
| 6 months | Cream | 96.9% | 79.8% | 31.2 mg/370 mg | 1.32% | 92% |
| | white | | | | | |

No significant differences were found in the values for gastro-resistence and release of active ingredient with respect to the initial values, independently of the storage conditions. Both tests were carried out according to Pharmacopea USP XXIII.

The power of active ingredient was determined by high-resolution liquid chromatography. The degradation products were evaluated bn the basis of the transmittance results detected at 440 nm.

From the results obtained it can be deduced that that there were no great differences with respect to the initial values. A slight loss of activity could be detected at six month's storage at a temperature of 40° C., which would explain the reduction of transmittance values at 440 nm.

The results obtained show the chemical stability of the active ingredient under the storage conditions tested. Moreover, no considerable variations in the humidity of the pellets were detected during storage, thus showing the physical stability of the formulation.

All these results show the stability of the formulations object of the present invention, which are moreover different from those described in the prior art in that they have no intermediate separating layer between the active layer and the gastro-resistant layer.

The electron scanning microscopy study was carried out using a Jeol JSM6400 scanning microscope. Photograph number 1 shows a section of the pellet of lansoprazol of example 1, showing clearly the presence of the inert nucleus, the active layer, intimately linked to the nucleus, and the gastro-resistant coating. Photographs numbers 2 and 3 show further details of both layers more clearly, revealing the absence of an intermediate separating layer between them. Photograph number 4 shows the low porosity of the coating. The lack of surface pores explains the physical-chemical stability of the pellet.

### Example 2

In a stainless steel receptacle the alcalizing aqueous solution of disodium phosphate was prepared, and to this were added the omeprazol, lactose and sodium lauryl sulphate. Agitation was maintained to total homogeneity and the colloidal solution of hydroxypropylmethyl cellulose (12.55% p/p) and hydroxypropyl cellulose (L-HPC) added. Agitation was maintained up till the moment of spraying onto the neutral pellets.

The qualitative-quantitative composition of the solution-suspension was as follows:

| | | |
|---|---|---|
| Omeprazol | 1.38 | Kg |
| Sodium lauryl sulphate | 5.28 10⁻³ | Kg |
| Chrystallized disodium phosphate | 0.052 | Kg |
| Hydroxypropylmethyl cellulose | 0.68 | Kg |
| Lactose | 0.51 | Kg |
| Hydroxypropyl cellulose | 0.39 | Kg |
| Water | 14.28 | Kg |

10 kg of inert nuclei was incorporated, made up of saccharose (62.5-91.5%) and starch (37.5-8.5%) of 800 micrometers average size in a NIRO “Wurster” type fluid bed and was covered with the solution-suspension prepared in advance, under the following conditions: air flow: 250 m³/hr. Diameter of nozzles: 1.2 mm. Spraying pressure: 2.5 bar. Spraying of product: 100 g/min. Air temperature: 75° C. Product temperature: 35° C.

The charged nuclei were then dried in order to obtain a suitable degree of humidity in the bed for 30 minutes with air at a temperature of 35° C. and with air flow of 250 m³/hr.

The dry granules were then subjected to enteric coating by spraying any of the gastro-resistant formulae shown below, prepared from the aqueous solution of polyethylene glycol to which were incorporated the other excipients under continuous agitation (Formula I) or from the organic solution of acetone and ethyl alcohol to which were incorporated the other excipients under continuous agitation (Formula II)

| Formula I | | |
|---|---|---|
| Talc | 0.57 | Kg |
| Titanium dioxide | 0.18 | Kg |
| Polyethylene glycol 6000 | 0.18 | Kg |
| Polysorbate | 0.08 | Kg |
| Eudragit L30D55 | 5.78 | Kg |
| Water | 12.14 | Kg |
| Formula II | | |
| Acetone | 20.86 | Kg |
| Hydroxypropylmethyl cellulose phthalate | 2.35 | Kg |
| Diethyl phthalate | 0.011 | Kg |
| Etyl alcohol | 8.93 | Kg |

For this purpose, work was carried out under the following conditions: air flow: 250 m³/hr. Diameter of nozzles: 1.2 mm. Spraying pressure: 2.5 bar. Spraying of product: 100 g/min. Air temperature: 70° C. Product temperature: 36° C.

The coated pellets were dried for 45 minutes with air at a temperature of 35° C. and with a flow of 250 m³/hr.

Below are set out the results of the stability studies carried out on a batch of Omeprazol under different storage conditions: ambient temperature, and 30° C. and relative humidity 65%.

| | | | | | | |
|---|---|---|---|---|---|---|
| | | Gastro- | | | | Transmittance |
| Test time | Colour | resistence | Release | Active Ing. | Humidity | at 440 nm |

Storage conditions: Ambient temperature
Container: Topaz glass bottle with bag of silica gel inside fitted with
metallic screw-threaded top including zelelastic seal

| | | | | | | |
|---|---|---|---|---|---|---|
| Zero hour | Cream | 99.0% | 94.0% | 20.4 mg/233 mg | 1.12% | 98% |
| | white | | | | | |
| 1 month | Cream | 99.6% | 93.7% | 20.5 mg/233 mg | 1.14% | 98% |
| | white | | | | | |
| 3 months | Cream | 98.9% | 93.5% | 20.6 mg/233 mg | 1.20% | 98% |
| | white | | | | | |
| 6 months | Cream | 98.6% | 93.0% | 20.3 mg/233 mg | 1.25% | 98% |
| | white | | | | | |
| 18 | Cream | 97.4% | 91.0% | 20.2 mg/233 mg | 1.35% | 96% |
| months | white | | | | | |

Storage conditions: Temperature: 30° C., 65% of humidity
Container: Topaz glass bottle with bag of silica gel inside fitted with
metallic screw-threaded top including zelelastic seal

| | | | | | | |
|---|---|---|---|---|---|---|
| Zero hour | Cream | 99.0% | 94.0% | 20.4 mg/233 mg | 1.12% | 98% |
| | white | | | | | |
| 1 month | Cream | 98.0% | 93.8% | 20.0 mg/233 mg | 1.16% | 97% |
| | white | | | | | |
| 3 months | Cream | 97.8% | 93.1% | 20.5 mg/233 mg | 1.26% | 96% |
| | white | | | | | |
| 6 months | Cream | 97.0% | 92.6% | 20.3 mg/233 mg | 1.37% | 95% |
| | white | | | | | |

The gastro-resistance, humidity and and release values explain the physical stability of the pellet under the storage conditions tested. For their part, the power of the active ingredient and the transmittance values at 440 nm ensure the chemical stability of the formulation.

All these results show the stability of the formulations object of the present invention, which moreover differ from those described in the prior art in that they have no intermediate separating layer between the active layer and the gastro-resistant layer.

The electron scanning microscopy study was carried out using a Jeol JSM6400 scanning microscope. Photographs numbers 5, 6 and 7 show a section of the pellet of omeprazol of example 2 with gastro-resistant coating of formula I, clearly showing the presence of the inert nucleus, the active layer, intimately linked to the nucleus, and the gastro-resistant coating. Photograph number 8 shows the homogeneity of the coating and the low number of pores, factors which enhance the physical stability of the pellet.

### Example 3

| | | |
|---|---|---|
| Omeprazol | 1.51 | Kg |
| Sodium lauryl sulphate | 2.20 10⁻² | Kg |
| Hydroxypropylmethyl cellulose | 1.09 | Kg |
| Lactose | 1.35 | Kg |
| Hydroxypropyl cellulose | 0.54 | Kg |
| Sodium acetate | 7.20 10⁻² | Kg |
| Water | 17.64 | Kg |

10 kg of inert nuclei, made up of saccharose (62.5-91.5%) and starch (37.5-8.5%) of 850 micrometers average size, were introduced and coated with the above mentioned solution-suspension under the following conditions:

Air flow: 5/72 m³/s

Diameter of nozzles: 1.2 mm

Spraying pressure: 2.5 10⁵ Pa

Spraying of product: 1/600 kg/s

Air temperature: 75° C.

Product temperature: 35° C.

The charged nuclei were then dried in the same bed for 45 minutes with air at a temperature of 35° C. and with air flow of 5/72.

The dry granules were then subjected to enteric coating by spraying any of the gastro-resistant solution-suspension detailed below.

| | | |
|---|---|---|
| Hydroxypropylmethylcellulose | 1.617 | Kg |
| acetate succinate (AS-MF) | | |
| Triethylcitrate | 0.45 | Kg |
| Talc | 0.48 | Kg g |
| Sorbitan sesquioleate | 4.04 10⁻⁴ | Kg |
| Water | 13.62 | Kg. |

The working conditions were as follows:

| Air flow: | 5/72 | m³/s |
|---|---|---|
| Diameter of nozzles: | 1.2 | mm |
| Spraying pressure: | 2.5 10⁵ | Pa |
| Spraying of product: | 1/600 | kg/s |
| Air temperature: | 70° | C. |
| Product temperature: | 35° | C. |

Drying of the coated pellets were carried out for 45 minutes at a temperature of 35° C. with an air flow of 5/72 m³/s.

The terms and expressions which have been employed are used as terms of description and not of limitation, and there is no intention in the use of such terms and expressions of excluding any equivalent of the features shown and described or portions thereof, it being recognized that various modifications are possible within the scope of the invention.

I claim:

1. An oral pharmaceutical preparation comprising:
a) an inert nucleus;
b) a soluble active layer or layer which disintegrates rapidly in water, made from a single aqueous or hydroalcoholic solution-suspension which comprises:
an active ingredient of anti-ulcer activity of general formula I 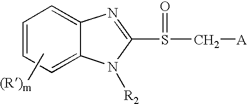
wherein:
A is: 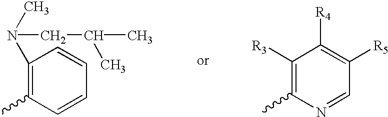
in which: R³ and R⁵ are the same or different, and may be hydrogen, alkyl, alkoxy, or alkoxyalkoxy;
R⁴ is hydrogen, alkyl, alkoxy which can optionally be fluorated, alkoxyalkoxy, or alkoxycycloalkyl;
R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulphinyl;
R² is hydrogen, alkyl, acyl, carboalkoxy, carbamoyl, alkylcarbamoyl, dialkylcarbamoyl, alkylcarbonilmethyl, alkoxycarbonilmethyl or alkylsulfonil; and, m is a whole number from 0 to 4;
or of formula II or III, 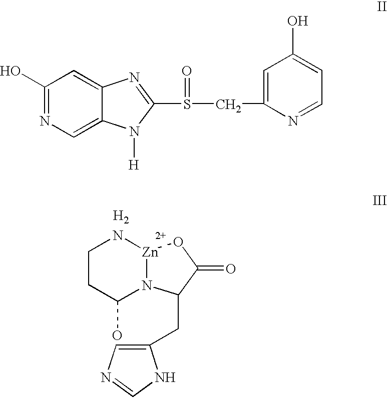
and
at least one pharmaceutically acceptable excipient selected from the group which includes: a binder, an alkaline reaction compound, a surface-active agent, a filling material and a disintegrating-swelling excipient; and
c) a gastro-resistant outer coating made from a solution which includes:
an enteric coating polymer; and
at least one excipient chosen from the group which includes: a plasticizer, a surface-active agent, a pigment and a lubricant.

2. The pharmaceutical preparation of claim 1, wherein the inert nucleus is a neutral spherical microgranule which includes in its composition at least two of: sorbitol, mannitol, saccharose, starch, microcrystalline cellulose, lactose, glucose, trehalose, maltitol or fructose.

3. The pharmaceutical preparation of claim 1, wherein the inert nucleus has an initial size between 200 and 1800 micrometers, preferably between 600-900 micrometers.

4. The pharmaceutical preparation of claim 1, wherein the binder in said aqueous or hydroalcoholic solution-suspension is selected from the group consisting of saccharose, starch, methyl cellulose, CMC, HPC, HPMC, polyvinyl pyrrolidone (PVP), dextrin or gum arabic, dissolved in water, ethanol, or a mixture of both at 50% (v/v).

5. The pharmaceutical preparation of claim 1, wherein the compound of alkaline reaction in said aqueous or hydroalcoholic solution-suspension is selected from the group consisting of trisodium phosphate, disodium phosphate, magnesium oxide, magnesium hydroxide, magnesium carbonate, aluminium hydroxide, carbonate, phosphate or citrate of aluminium, calcium, sodium or potassium, the mixed compounds of aluminium/magnesium Al₂O₃.6MgO.CO₂.12H₂O or MgO.Al₂O₃.2SiO₂.nH₂O and amino acids with alkaline reaction.

6. The pharmaceutical preparation of claim 1, wherein the surface-active agent in said aqueous or hydroalcoholic solution-suspension is selected from the group consisting of sodium lauryl sulphate, polysorbate, poloxamer or other ionic and non-ionic surface-active agents.

7. The pharmaceutical preparation of claim 1, wherein said filling material in said aqueous or hydroalcoholic solution-suspension is selected from the group consisting of lactose, starch, saccharose and microcrystalline cellulose.

8. The pharmaceutical preparation of claim 1, wherein said disintegrating-swelling excipient in said aqueous or hydroalcoholic solution suspension is selected from the group consisting of starch, CMCCa, sodium glycolate starch and L-HPC.

9. The pharmaceutical preparation of claim 1, wherein said enteric coating polymer in said external gastro-resistant coating is selected from the group consisting of methyl cellulose, HEC, HBC, HPMC, ethyl cellulose, HMC, HPC, polyoxyethylene glycol, castor oil, cellulose phthalic acetate, phthalate of HPMC, succinate acetate of HMC, sodium carboxymethylamylopectin, chitosan, alginic acid, carrageenans, galactomannons, tragacanth, shellac, agar-agar, gum arabic, guar gum, xanthan gum, polyacrylic acids, methacrylics and their salts, PVA, polyethylene and polyproprylene oxides and mixtures thereof.

10. The pharmaceutical preparation of claim 1, wherein said plasticizer in said external gastro-resistant coating is selected from the group consisting of TEC, PEG, cetyl alcohol and stearyl alcohol.

11. The pharmaceutical preparation of claim 1, wherein said surface-active agent present in said external gastro-resistant coating layer is selected from the group consisting of sodium lauryl sulphate, polysorbate and poloxamer.

12. The pharmaceutical preparation of claim 1, wherein said pigment in said external gastro-resistant coating layer is selected from the group consisting of titanium dioxide and iron sesquioxide.

13. The pharmaceutical preparation of claim 1, wherein said lubricant in said external gastro-resistant coating layer is selected from the group consisting of talc, magnesium stearate and glyceryl monostearate.

14. A process for making an oral pharmaceutical preparation comprising:
a) coating inert nuclei to form a layer thereon by spraying aqueous or hydroalcoholic suspension-solution, which comprises:
an active ingredient of anti-ulcer activity of general formula I wherein:
A is: 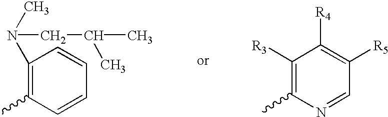
wherein R³ and R⁵ are the same or different, and may be hydrogen, alkyl, alkoxy, or alkoxyalkoxy;
R⁴ is hydrogen, alkyl, alkoxy which can be fluorated, alkoxyalkoxy, or optionally alkoxycycloalkyl;
R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulphinyl;
R² is hydrogen, alkyl, acyl, carboalkoxy, carbamoyl, alkylcarbamoyl, dialkylcarbamoyl, alkylcarbonilmethyl, alkoxycarbonilmethyl or alkylsulfonil; and, m is a whole number from 0 a 4;
or general formula II or III, 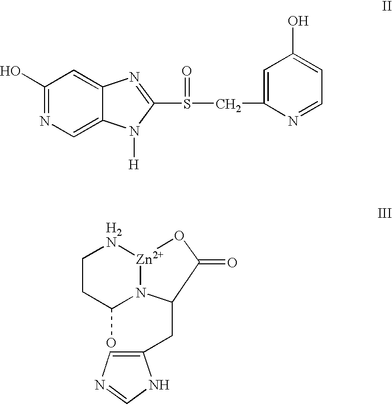
and
at least one pharmaceutically acceptable excipient selected from the group which includes: a binder, an alkaline reaction compound, surface-active agents, a filling material and disintegrating-swelling excipients;
b) drying the active layer formed during said spraying to form charged nuclei; and
c) coating the charged nuclei by spraying a solution which contains an enteric coating polymer with at least one pharmaceutically acceptable excipient selected from the group comprising: a plasticizer, a surface-active agent, a pigment and a lubricant, to form an gastro-resistant external coating layer.

15. The process of claim 14 further comprising drying the coated charged nuclei.

16. The process of claim 14, wherein said binder in said aqueous or hydroalcoholic solution-suspension is selected from the group consisting of saccharose, starch, methylcellulose, CMC, HPC, HPMC, polyvinyl pyrrolidone (PVP), dextrin or gum arabic, either alone or mixed, dissolved in water, ethanol or a mixture of both at 50% (v/v).

17. The process of claim 14, wherein said compound of alkaline reaction in said aqueous or hydroalcoholic solution-suspension is selected from the group consisting of trisodium phosphate, disodium phosphate, magnesium oxide, magnesium hydroxide, magnesium carbonate, aluminium hydroxide, carbonate, phosphate or citrate of aluminium, calcium, sodium or potassium, and the mixed compounds of aluminium/magnesium Al₂O₃.6MgO.CO₂.12H₂O or MgO.Al₂O₃.2SiO₂.nH₂O and amino acids with alkaline reaction.

18. The process of claim 14, wherein said surface-active agent present in said aqueous or hydroalcoholic solution-suspension is selected from the group consisting of sodium lauryl sulphate, polysorbate, poloxamer or other ionic and non-ionic surface-active agents.

19. The process of claim 14, wherein said filling material in said aqueous or hydroalcoholic solution-suspension is selected from the group consisting of lactose, starch, saccharose and microcrystalline cellulose.

20. The process of claim 14, wherein said disintegrating-swelling excipient in said aqueous or hydroalcoholic solution-suspension is selected from the group consisting of starch, CMCCa, sodium glycolate starch and L-HPC.

21. The process of claim 14, wherein said enteric coating polymer in said external gastro-resistant coating is selected from the group consisting of methyl cellulose, HEC, HBC, HPMC, ethyl cellulose, HMC, HPC, polyoxyethylene glycol, castor oil, cellulose phthalic acetate, phthalate of HPMC, succinate acetate of HMC, sodium carboxymethylamylopectin, chitosan, alginic acid, carrageenans, galactomannons, tragacanth, shellac, agar-agar, gum arabic, guar gum, xanthan gum, polyacrylic acids, methacrylics and their salts, PVA, polyethylene and polyproprylene oxides and mixtures thereof.

22. The process of claim 14, wherein said plasticizer in said external gastro-resistant coating is selected from the group consisting of TEC, PEG, cetyl and stearyl alcohol.

23. The process of claim 14, wherein said surface-active agent in said aqueous or hydroalcoholic solution-suspension is selected from the group consisting of sodium lauryl sulphate, polysorbate and poloxamer.

24. The process of claim 14, wherein said pigment in said external gastro-resistant coating layer is selected from the group consisting of titanium dioxide and iron sesquioxide.

25. The process of claim 14, wherein said lubricant in said external gastro-resistant coating layer is selected from the group consisting of talc, magnesium stearate and glyceryl monostearate.

26. The pharmaceutical preparation of claim 1 wherein the filling material is selected from the group consisting of mannitol, sorbitol or gelatin.

27. The pharmaceutical preparation of claim 1 wherein the alkaline reacting compound is selected from the group consisting of sodium, potassium, aluminum or calcium acetate; sodium, potassium, aluminum or calcium glycerophosphate; (tris)-hydroxymethylaminemethane (tromethamine); N-methylglucamine, 2-amine-2-methyl-1,3-propanediol; 2-amine-2-methyl-1propanole; sodium, potassium, magnesium, calcium, aluminum or aluminum hydroxide salts of aminoacids like lysine, glutamic acid, glycine or pyrimidinecarboxilic acids, like nicotinic acid, salts derived from organic or weak inorganic acids and bases like guanidine and basic aminoacids like arginine, histidine, lysine and triptophane.

28. The pharmaceutical preparation of claim 1 wherein the enteric coating polymer is selected from the group consisting of HPMC acetate succinate, polyvinyl acetate phthalate, and cellulose acetate trimethylate.

29. The pharmaceutical preparation of claim 1 wherein the plasticizer is selected from the group consisting of diethyl phthalate, dibutyl phthalate, dimethyl phthalate, diocytl adipate, dioctyl phthalate, dioctyl terephthalate, butyloctyl phthalate, triethylene glycol di-2-ethylhexanoate, trioctylmethylate, glyceryl triacetate, glyceryl tripropionate, 2,2,4-trimethyl-1,3-pentanedioldiisobutyrate.

30. The process of claim 14 wherein filling materials is selected from the group consisting of mannitol, sorbitol or gelatin.

31. The process of claim 14 wherein the alkaline reacting compound is selected from the group consisting of sodium, potassium, aluminum or calcium acetate; sodium, potassium, aluminum or calcium glycerophosphate; (tris)-hydroxymethylaminemethane (tromethamine); N-methylglucamine, 2-amine-2-methyl-1,3-propanediol; 2-amine-2-methyl-1propanole; sodium, potassium, magnesium, calcium, aluminum or aluminum hydroxide salts of aminoacids like lysine, glutamic acid, glycine or pyrimidinecarboxilic acids, like nicotinic acid, salts derived from organic or weak inorganic acids and bases like guanidine and basic aminoacids like arginine, histidine, lysine and triptophane.

32. The process of claim 14 wherein the enteric coating polymer is selected from the group consisting of HPMC acetate succinate, polyvinyl acetate phthalate and, cellulose acetate trimethylate.

33. The process of claim 14 wherein the plasticizer is selected from the group consisting of diethyl phthalate, dibutyl phthalate, dimethyl phthalate, diocytl adipate, dioctyl phthalate, dioctyl terephthalate, butyloctyl phthalate, triethylene glycol di-2-ethylhexanoate, trioctylmethylate, glyceryl triacetate, glyceryl tripropionate and, 2,2,4-trimethyl-1,3-pentanedioldiisobutyrate.
